(19) United States
(12) Patent Application Publication
(10) Pub. No.: US 2012/0143081 A1
(43) Pub. Date: Jun. 7, 2012

(54) Pure silver device and method for diagnosing and treating pain in the human body

(76) Inventor: Hyung Woo Lyu, Harrington Park, NJ (US)

(21) Appl. No.: 12/928,204

(22) Filed: Dec. 6, 2010

(51) Int. Cl. A61B 5/00; A61H 39/04

(52) U.S. Cl. 600/557; 601/135

(57) ABSTRACT

Disclosed herein are a pure silver device and a method for diagnosing and treating pain on human body. The pure silver device comprises an elongated handle, a head having both a hammer head and a comb opposed to each other, a panel, and an outwardly extending protrusion on the bottom end of the handle. The pure silver device is used to diagnose and treat pain by finding out pressure pain points on both abdominal area, at which root of pain is located, and a symptomatic area of the human body and then by releasing the pressure pain points by tapping, pressing, rubbing, combing or combining the foregoing. Owing to the highest thermal and electrical conductivity of silver, the pure silver device produces amazingly better stimulation of the pressure pain points. Therefore, the pure silver device is more effectively used for diagnosing and treating pain than other devices made out of any other material, even sterling silver and magnetic material. In addition, the pure silver device is portable and useful in that anyone can diagnose and treat himself or herself anytime and anyplace. The pure silver device and the method are simple, easy, safe without any side effects and medical risks, and economical.

### BACKGROUND OF THE INVENTION

1. Field of the Invention

The present invention relates to a method and a pure silver device for diagnosing and treating pain in the human body.

2. Description of Related Prior Art

Many people suffer from various kinds of pain in particular areas of their bodies. The pain can be treated by traditional eastern medicine such as acupuncture, moxibustion, and acupressure.

In traditional Eastern medicine, pressure pain points are spots on the body where there is pain on the skin. This usually occurs when a certain amount of pressure is applied. Pressure pain points represent physical problems due to a nerve impulse, blood flow, or the stagnation of Qi, regarded as “bio-energy flow”. Most of bodily pain and diseases start with stagnation of blood and Qi. If someone has stagnation in the abdominal area, called the ‘root of life’ or ‘hara’ in Japanese, and/or in the distal areas (the head and the four limbs), he or she cannot get enough energy in certain parts of the body, and this creates a problem. Therefore, Eastern medicine is aimed at not only achieving harmonious flow of blood and Qi in the meridian channels, which are connected points across the anatomy which affect a specific organ or other part of the body, but also releasing the stagnation with acupuncture, moxibustion, herbs, or acupressure therapy.

Acupuncture is an Eastern healing art using needles to be inserted into key points (acupuncture points) on various parts of the human body, which stimulate the body's natural self-curative abilities. However, acupressure is performed by using the fingers and other parts of the body to skillfully press the key points. When these trigger points are pressed, they release muscular tension, promote circulation of blood, and release the body's life force energy to aid healing. Acupuncture and acupressure use the same pressure points and meridians, but acupuncture employs needles, while acupressure uses gentle to firm pressure and integrates bodywork therapies, energy psychology, and massage therapy techniques. Acupressure therapy has advantages including relieving stress and pain, balancing the body, and maintaining good health. By relieving stress, acupressure therapy strengthens resistance to disease and promotes wellness. Generally, unlike acupressure therapy, acupuncture therapy has known side effects including harm to internal organs, infection due to improper sterilization of the needles, weakening of the human body's system due to the reaction of needles, and bleeding in the needle inserted areas.

Many Eastern medicine practitioners have been looking into using non-invasive, effective, pain-free materials for treatment. Examples include magnets, stones, gold, silver, aluminum, titanium, and electrically stimulating tools.

The therapeutic effect of silver has been partially utilized for thousands of years in different cultures such as an antibiotic to prevent bacterial and fungal infection without high toxicity to humans, but there has been no systematic approach to utilize its beneficial effect on health.

The inventor has invented and used pure silver rings not only to assess both the personal constitution of an individual and whether the flow of bio-energy within the body is normal or obstructed but also to harmonize the obstructed bio-energy. One year ago, the inventor published the method to use pure silver rings in local newspapers and local broadcasts in New Jersey, USA and on the internet.

### SUMMARY OF THE INVENTION

The invention is a pure silver device and a method for diagnosing and treating pain in the human body. The pure silver device as a whole is made out of at least 99.9% fine silver. The pure silver device comprises an elongated handle, a head having both a hammer head and a comb opposed to each other, a panel, and an outwardly extending protrusion on the bottom end of the handle. Said panel is vertically attached either between said bottom end and said outwardly expending protrusion or to a point of about 1 to 3 cm away from said bottom end.

The pure silver device is used to diagnose pain by finding out pressure pain points on both the abdominal area, at which the root of pain is located, and symptomatic areas of the human body by pressing both areas with said outwardly extending protrusion of the device. In addition, the pure silver is used to treat pain firstly by releasing the pressure pain points on abdominal area by tapping with either said hammer head itself or both said hammer and a pure silver nail which has no sharp protrusion, and secondly by releasing the pressure pain points on symptomatic areas of the human body by tapping with said hammer head, both said hammer and the pure silver nail together, said panel, or said comb, by pressing and/or rubbing with said panel, said comb or said outwardly extending protrusion, by combing on the scalp of the head of the body with said comb, or combining the foregoing. The tapping on the pressure points with said hammer head of the pure silver device is performed along with the pure silver nail which has no sharp protrusion on its the bottom by putting said pure silver nail vertically on the skin of the pressure pain points and then tapping on the upper top surface of said pure silver nail with said hammer head. In this case of tapping with both said hammer head and the pure silver nail together, the treatment of pain is more effective than tapping with only said hammer head because the friction force generated from tapping with said hammer head on the top of the pure silver nail can produce more electric power and strengthen the flow of electricity into the body.

Because silver has the highest thermal conductivity of any element and the highest electrical conductivity of any metal, silver promotes the stimulation of the pressure pain points. Pure silver effectively maximizes the highest thermal and electrical conductivity of silver. The pure silver device produces better stimulation of the pressure pain points without penetration into the skin of the human body. Therefore, the pure silver device is more effectively used for diagnosing and treating pain than other devices made out of any other material, even sterling silver and magnetic material. Using abdominal diagnosis based on the basic theory of eastern medicine, the pure silver device and the method carry out the diagnosis and treatment of the root of pain on the abdomen which fundamentally causes and/or is connected to the complained-of pain and symptoms, and thus the effect of treatment can be positively maximized, and the recurrence of pain can be prevented.

In addition, the pure silver device is portable, monetarily valuable for personal property, and durable for the treatment of pain. The pure silver device and the method are simple, easy, and useful in that anyone can diagnose and treat themselves anytime and anyplace as well as without even the knowledge of basic Eastern medicine such as meridians and acupuncture points. Due to the antibiotic effect of silver itself as well as no penetration into the skin, the pure silver device and the method are very safe without any risk of infections, side effects, or medical risks, so even children and seniors get safely treated.

### BRIEF DESCRIPTION OF THE DRAWINGS

FIG. 1 shows an acupuncture channel and points on the chest and abdomen.

FIG. 2A is a perspective view of the preferred embodiment of the pure silver device.

FIG. 2B is a top view of the preferred embodiment of the pure silver device.

FIG. 2C is a bottom view of the preferred embodiment of the pure silver device.

FIG. 2D is a front elevational view of the preferred embodiment of the pure silver device.

FIG. 2E is a right side elevational view of the preferred embodiment of the pure silver device.

FIG. 3 shows the direction of abdominal diagnosis for individuals who have no knowledge of acupuncture points on the abdomen of the human body.

FIG. 4 shows use of the outwardly extending protrusion of the pure silver device to diagnose pain on the abdomen of human body.

FIG. 5 shows use of both the hammer head of the pure silver device and a pure silver nail together to treat pain on the abdomen of the human body.

FIGS. 6A-6E show use of the preferred embodiment of the pure silver device to treat pain on other parts of the human body than the abdomen.

### DETAILED DESCRIPTION OF THE INVENTION

According to Eastern medicine such as acupuncture, acupressure in China, Korea, and Japan, pain and symptoms of diseases and/or disorders are mostly the result of disharmony of the function of the human body due to disruption or blocking of bio-energy flow and/or blood circulation within the body. From an electrical viewpoint, the human body is a very weak conductor, but inside our bodies, there are many electrically good-conducting channels called the meridian channels. Bio-energy flows through these channels and supplies the energy where needed. When the flow is not inhibited by stagnation, one is in a healthy condition. But when stagnation occurs in an area, a large resistance exists there, and this causes sparks or pain in that area because bio-energy cannot go through easily. Thus, the channel in that area is damaged or partially blocked. The inventor shows that placing silver, which has the highest electrical and thermal conductivities, at the stagnation area is like ‘shorting’ the damaged channel by an external conductor. By tapping, pressing, or rubbing with silver in the stagnation area, the bio-energy flow is restored and the body's self-repair begins. In time, this external restoration enhances the repair of the damaged channel. After some period of treatments, the repair is finished, and the stagnation is eliminated.

Although there are several hundreds of acupuncture points on human body, the acupuncture points on the skin of abdominal area are the significant points for the diagnosis and treatment of pain and diseases in that the bio-energy of internal organs in the body aggregate at or around the acupuncture points on the abdomen. Specific acupuncture points on the abdomen are interrelated with to bio-energy and function of specific internal organs, which are called viscerosomatic reflexes. The pressure pain points on the abdominal area mostly correspond with the acupuncture points on the abdomen that indicate that the flow of bio-energy of a particular internal organ is obstructed or blocked, which causes unbalance of the function of the organ as well as the whole body. Moreover, unless the pressure pain points on the abdomen are released, in other words, if the pressure pain on the abdomen is not treated, it will cause other pain or diseases in the body. The pain on the abdomen is called the root of pain in the terms of Eastern medicine. Most individuals cannot normally feel pain in the abdomen without the procedure of pressing with force on skin of the abdomen. Thus, it is very important to diagnose and treat the pressure pain on the abdomen at which the root of pain is located, before the complained-of pain in the symptomatic area of the body is diagnosed and treated.

Abdominal diagnosis of pain and diseases with acupuncture channels and points on the abdomen had been systematically developed in Korea and Japan in that both countries, produced their own established acupuncture channel and points for abdominal diagnosis called Koryo Hand Therapy's Abdominal Diagnosis and Kiiko's Hara Diagnosis respectively, while in China, simple abdominal palpation had been performed for physical examination of pain and symptoms by determining painful areas and its firmness and by qualifying pain felt by the patient. Both Koryo Hand Therapy's Abdominal Diagnosis and Kiiko's Hara Diagnosis also include a couple of acupuncture points on the chest of the body. Although there are some different acupuncture points between Koryo Hand Therapy's Abdominal Diagnosis and Kiiko's Hara Diagnosis, most acupuncture points for abdominal diagnosis between them are same. Accordingly, the two abdominal acupuncture points can be easily combined and used together.

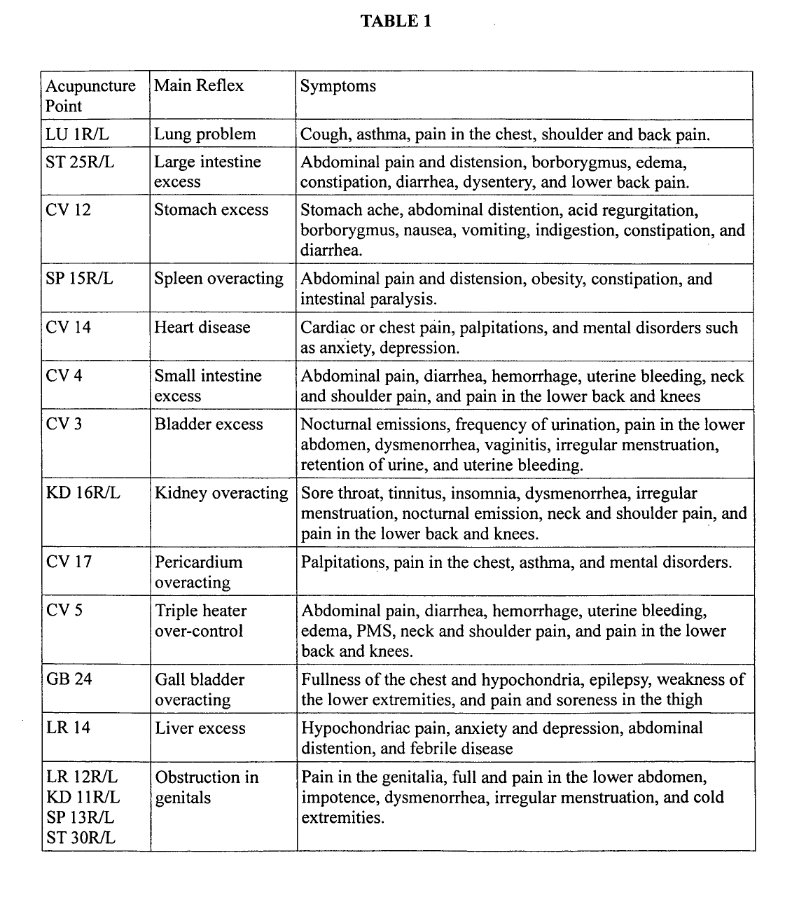

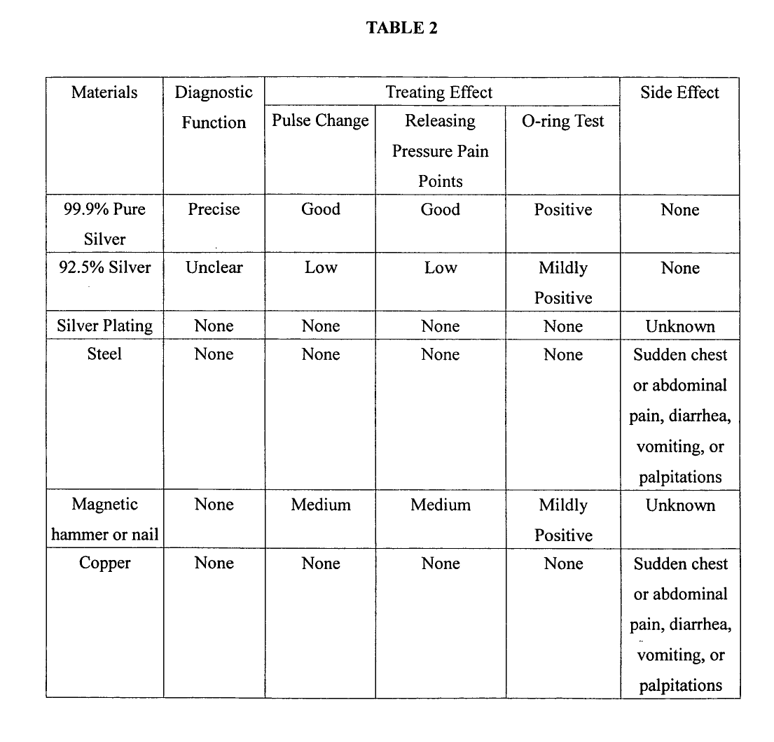

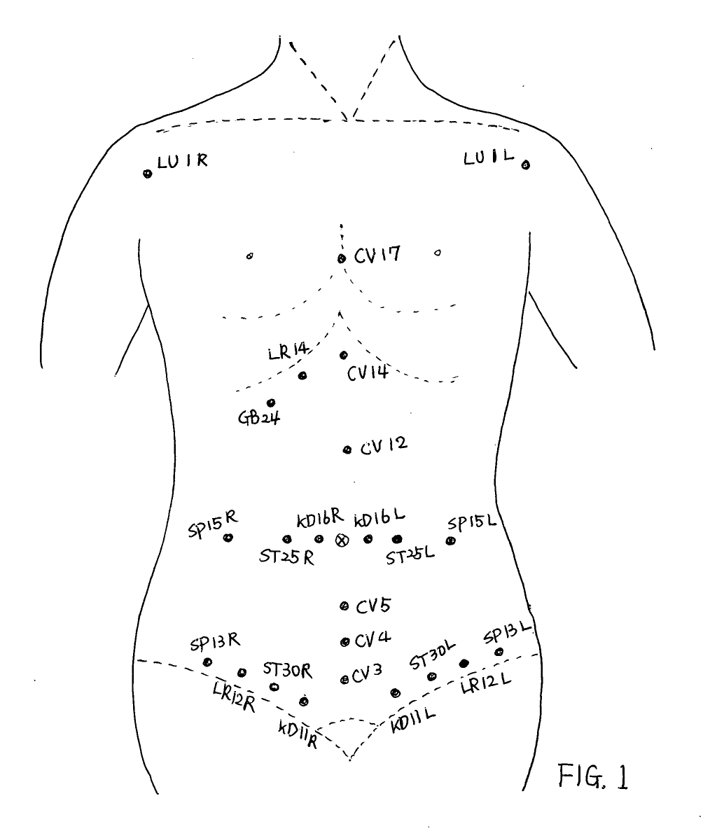

FIG. 1 shows the combined acupuncture channel and points for abdominal diagnosis, which illustrates significant fifteen acupuncture points on the chest and abdomen (LU1, ST25R/L, CV12, SP15R/L, CV14, CV4, KD16R/L, CV17, CV5, GB24, LR14, LR12R/L, KD11R/L, SP13 R/L, and ST30R/L). The acupuncture points on the abdomen of the human body are related to reflexes and symptoms. If an individual feels pain with pressure on the acupuncture points on his or her abdomen, he or she can diagnose which internal organ has a problem and what symptoms are occurring or could occur. Table 1 summarizes the main reflex and symptoms of pressure pain on the acupuncture points on the abdomen shown on FIG. 1.

The invention is a pure silver device and a method for diagnosing and treating pain in the human body. The invention is new systematic “theragnosis” applying pure silver to oriental medicine as alternative and preventative medicine. The invention is directed to using the pure silver device instead of acupuncture needles, sticks and fluff for moxibustion, or cups for cupping to stimulate the skin in order to get the same effect as or similar to acupuncture, moxibustion, or cupping, but without any infections or side effects. The inventor has identified that human pain is more effectively treated with a pure silver device by providing treatments not only directly onto pressure pain points on complained-of symptomatic areas of the body, but also onto pressure pain points on the abdominal area of the body at which the root of the complained-of pain is located. The root of the pain fundamentally causes and/or is connected to the complained-of pain and symptoms and could cause other pain and symptoms. Thus, along with the complained-of pain and symptoms, the root of pain on the abdomen of the body should be treated. If an individual has knowledge of the acupuncture points on the abdomen of the human body, he or she will easily find out the pressure pain points by pressing with force on the skin of his or her abdomen according the acupuncture points. The invention shows that even though an individual does not have any knowledge of acupuncture points on the abdomen of human body, he or she can by himself or herself find out the acupuncture points on his or her abdomen where individuals feel pain pressing with force on the skin of all around his or her abdominal area, which ranges from above the pelvis to below the ribs, inch by inch on all outward directions from the navel, because the pressure pain points on his or her abdomen mostly correspond with the acupuncture points on the abdomen of human body. Moreover, even though the pressure pain points on the abdomen of an individual do not conform to the acupuncture points on the abdomen of the human body, the pressure pain points should be released because pressure pain on the abdomen is regarded as the presence of diseases or medical problems in the body.

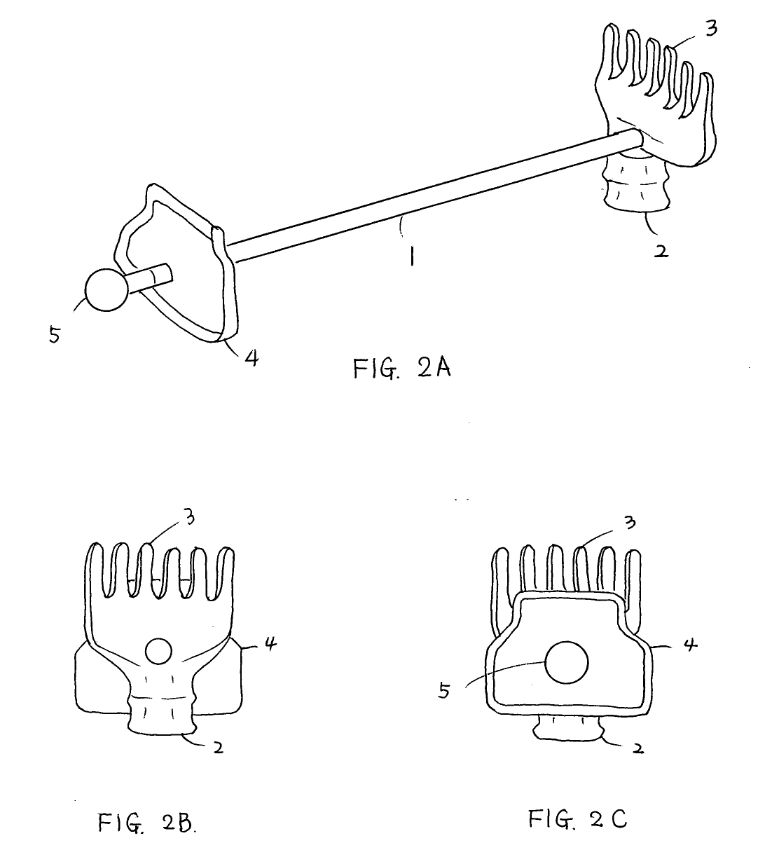

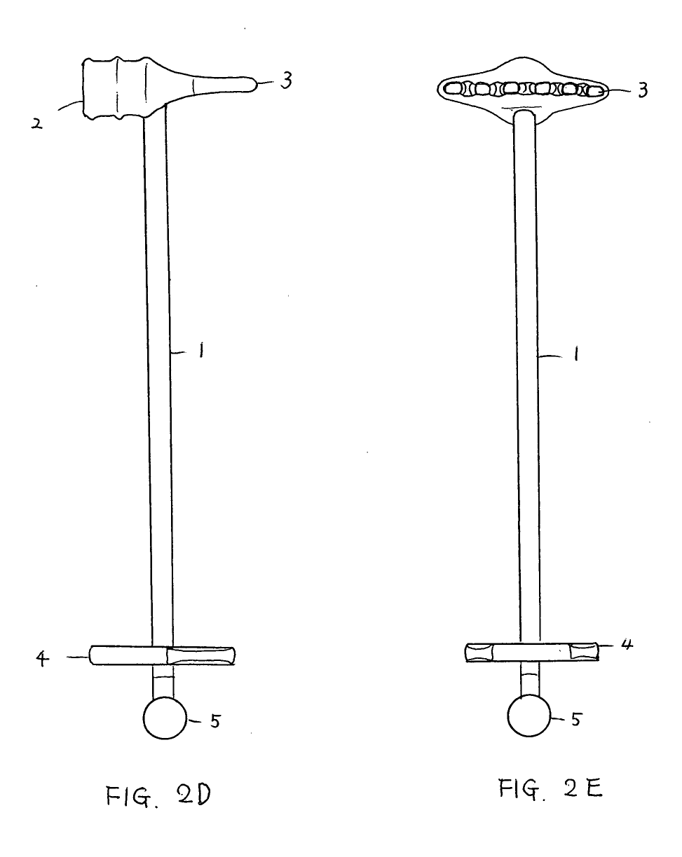

The pure silver device as a whole is made out of at least 99.9% fine silver. FIG. 2 is a drawing of the the preferred embodiment of the pure silver device. A is a perspective view of the pure silver device. B is a top view of the pure silver device. C is a bottom view of the pure silver device. D is a front elevational view of the pure silver device. E is a right side elevational view of the pure silver device. The preferred embodiment of the pure silver device comprises an elongated body portion which forms a handle (1), a head having both a hammer head (2) and a comb (3) opposed to each other, a panel (4), and an outwardly extending protrusion (5) on the bottom end of the handle. The handle (1), which is preferably about 15-22 cm long and preferably of a cylindrical shape with diameter of about 6-10 mm, has a top end and a bottom end. The head, which is preferably about 4-5 cm long, connected to said top end has a first end and a second end. Said first end is shaped to form a hammer head (2), having no sharp protrusion, and said second end is shaped to form a comb (3) having teeth. The hammer head (2) and the comb (3) are opposed to each other. The hammer head (2) is preferably of a cylindrical shape with a diameter of about 10-20 mm. The comb (3) has preferably about 5-7 teeth and is preferably 3-4 cm wide. Said bottom end has an outwardly expending protrusion (5) which is preferably of a sphere shape with a diameter of about 10-15 mm. The outwardly expending protrusion (5) can be in the form of a bead, an egg, a rounded mass, a nipple, a lump, a bump, a projection, or any combination thereof. The panel (4) is vertically attached between said bottom end and said outwardly expending protrusion. Preferably, the panel (4) is vertically attached to the point of about 1 to 3 cm away from said bottom end. The panel (4) is preferably about 2-3 mm thick, about 3.5-5 cm wide, and about 2.5-3.5 cm high. It is also in the unique shape of having both corners of the top of the rectangle indented roundly so that the length of the top edge or face is preferably 2-3 cm. Other panel shapes include, but are not limited to, triangle, rectangle, trapezoid, ellipse, semicircle, quarter-circle or any combination thereof.

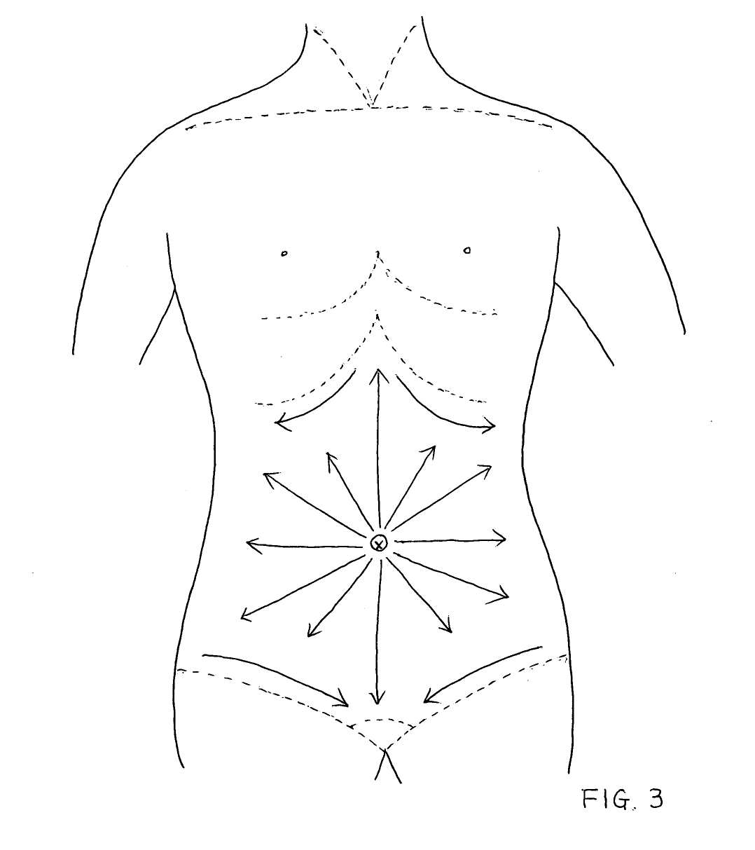

The pure silver device is used to diagnose pain by finding out pressure pain points on the skin of both abdominal area for root of pain and a symptomatic area of human body by pressing both areas with said outwardly extending protrusion (5) of the device. In order to detect the pressure pain points on the abdomen of the body, the outwardly extending protrusion of the device (5) is used to press either on the skin of the acupuncture points on the abdomen as shown on FIG. 1 or on the skin all around the abdominal area, which ranges from above the pelvis to below the ribs, inch by inch on all outward directions from the belly button as shown on FIG. 3, unless an individual has the knowledge of the acupuncture points on the abdomen. When the individual by himself or herself performs the abdominal diagnosis by pressing with the outwardly extending protrusion of the device on the skin of all around abdominal area, he or she can mark with tape or pen on all of the detected pressure pain points on the abdomen in order to recognize easily where the pressure pain points are located for the treatment of the pressure pain points. FIG. 4 shows use of the outwardly extending protrusion (5) of the pure silver device by either an individual, such as a trained professional, or a patient by himself or herself to apply pressure to the skin of the abdomen of the patient. When pressing with the outward extending protrusion (5) on the skin of the abdomen, the method of pressing includes the steps of a) applying a gentle force (first level force) on a particular point of the skin of the abdomen; b) if pressing with a gentle force there is no pain on the point, then applying a medium force (second level force) on the point; c) if pressing with the medium force there is no pain on the point, then applying a firm (third level force) on the point; and d) if pressing with the firm force there is no pain on the point, then moving either by an inch onto another point of the skin or onto another acupuncture point on the abdomen, and then the three steps of pressing are performed on the another point. The firm force means to press deep in the skin of the point. This procedure should continue until the skin of the entire area of the abdomen is entirely pressed through the method of the above-mentioned steps. In the case that the individual feels pain when either pressing with the gentle force on the point or the pressing with the medium force on the point, the pressing should be stopped and moved onto another point. When detecting the pressure pain points on the symptomatic area of the body, like the steps of pressing on the abdomen, applying three different level forces in a gentle, medium, and firm force order with the outwardly extending protrusion (5) of the device on the skin of the symptomatic area of the body. The point of pain as a pressure pain point should be recorded with which level force of pressing was used to detect the point of pain in order to effectively treat pain on the point. Along with the pure silver device to diagnose pain, pure silver rings with many different sizes for fingers can be used to find easily out both personal constitution of the individual and whether the flow of bio-energy within the body is normal or obstructed by putting the pure silver rings on each finger from thumb to little finger of left and/or right hand and then by taking both a pulse on the wrist and Bi-Digital O-ring Test that is an alternative medicine procedure in which the individual forms an ‘O’ with his or her fingers and the diagnostician subjectively evaluates the individual's finger strength as he or she tries to pry them open with his or her own fingers.

Also, the pure silver is used to treat pain firstly by releasing the pressure pain points on the skin of the abdominal area by tapping with either the hammer head (2) itself or both the hammer head (2) and a pure silver nail along together which has no sharp protrusion, and secondly by releasing the pressure pain points on the skin of the symptomatic areas of human body by tapping with the hammer head (2), both the hammer head (2) and the pure silver nail together, the comb (3), or the panel (4) by pressing with the comb (3), the panel (4), or the outwardly extending protrusion (5), by rubbing with the comb (3), the panel (4) or the outwardly extending protrusion (5), by combing on the scalp of the head of the body with the comb (3), or combining the foregoing. The tapping on the pressure points with the hammer head (2) of the pure silver device is performed along with the pure silver nail which has no sharp protrusion on its bottom by putting the pure silver nail vertically on the skin of the pressure pain points and then tapping on the upper top surface of the pure silver nail with the hammer head (2). FIG. 5 shows use of both the hammer head (2) of the pure silver device and the pure silver nail together by either an individual, such as a trained practitioner, or a patient by herself or himself to tap on the pressure pain points of the patient's abdomen. In this case of tapping with both said hammer head (2) and the pure silver nail together, the treatment of pain is more effective than tapping with only the hammer head (2) because the friction force generated from tapping with said hammer head (2) on the top of the pure silver nail can produce electricity and promote the flow of electricity into the body.

When tapping with either the hammer head (2) only or the hammer head (2) and the nail together on the pressure pain points on the abdomen and symptomatic area of the body to release all pressure pain points, in the case of the pressure pain points detected with a gentle force pressing, the strength of the tapping is either weak or medium so that a patient cannot feel uncomfortable and sore; the speed of the tapping is about 60 to 70 times a minute; and the tapping is performed for about 2 to 3 minutes. After that, if the pain on the pressure pain points still remains, more tapping is necessary so that the tapping is performed about 3 to 4 more times. Even though the frequency of the tapping is increased and the duration of the tapping is long due to the severity of pain, there are no side effects at all. In the case of pressure pain points detected with a medium force pressing, the strength of the tapping is either medium or strong in a way that a patient can feel positively fine; the speed of the tapping is about 60 to 70 times a minute; and the tapping is performed for about 1 to 2 minutes. After that, if the pain still remains, the tapping is performed about 2 to 3 more times. In the case of the pressure pain points detected with a firm force pressing, the strength of the tapping is either medium or strong; the speed of the tapping is about 60 to 70 times a minute; and the tapping is performed for 1 minute. After that, if the pain still remains, the tapping is performed about 1 to 2 more times. In addition, either the comb (3) or the panel (4) is used to tap on the pressure pain points on the symptomatic areas other than the abdomen with the same tapping method mentioned above.

After the pressure pain points on both the abdomen and the symptomatic area of the body are released through the tapping techniques, pressing or rubbing with either the comb (3) or the panel (4) on and around the pressure pain points is much helpful for a patient to feel better in promoting both the flow of bio-energy and the circulation of the blood obstructed by the pain and symptoms. When rubbing, an individual preferably uses either teeth of the comb (3) or the edge or face of the width of the panel (4).

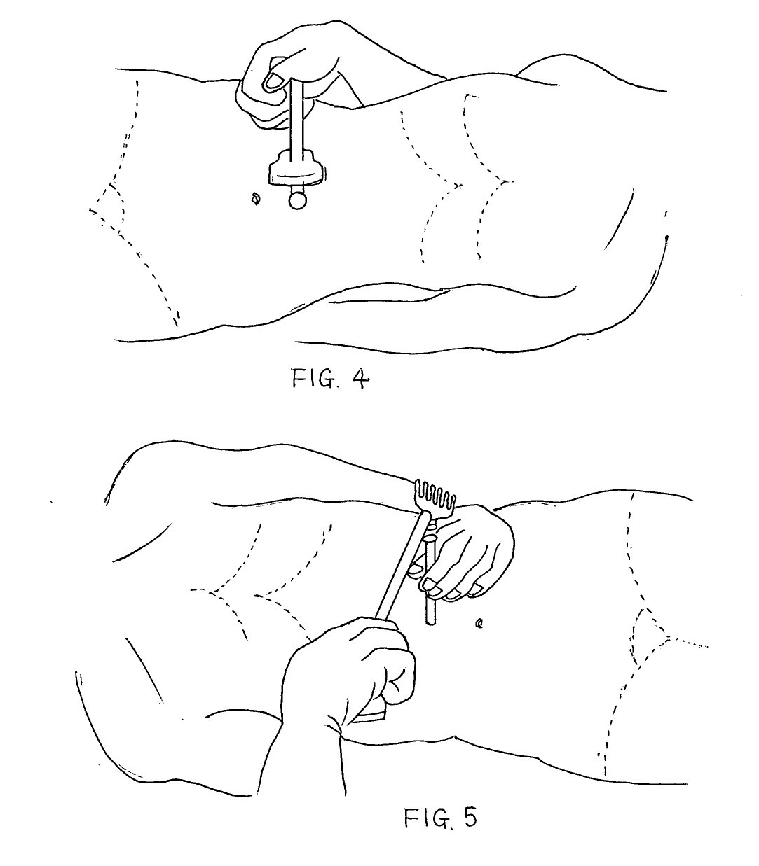

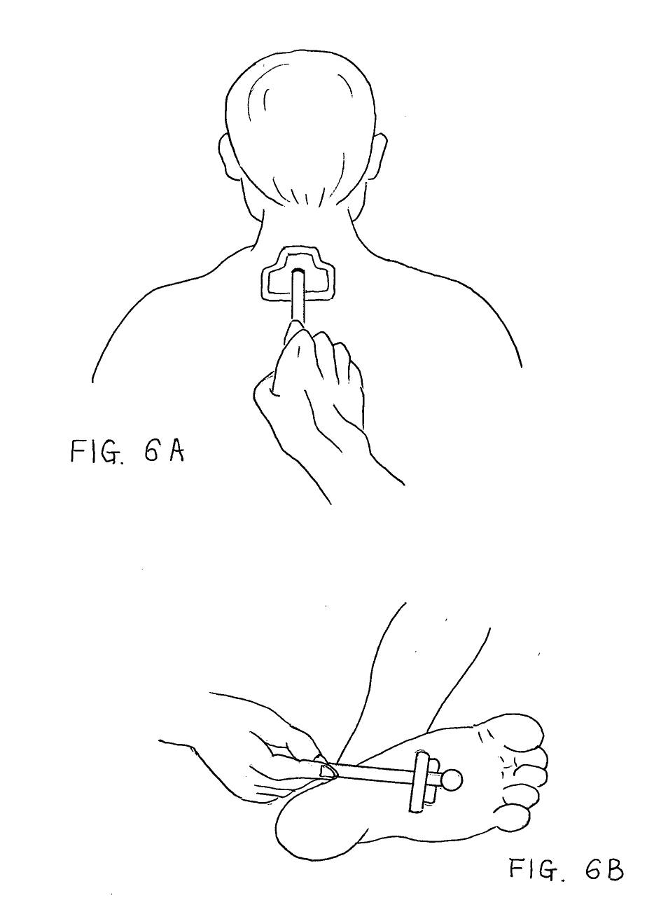

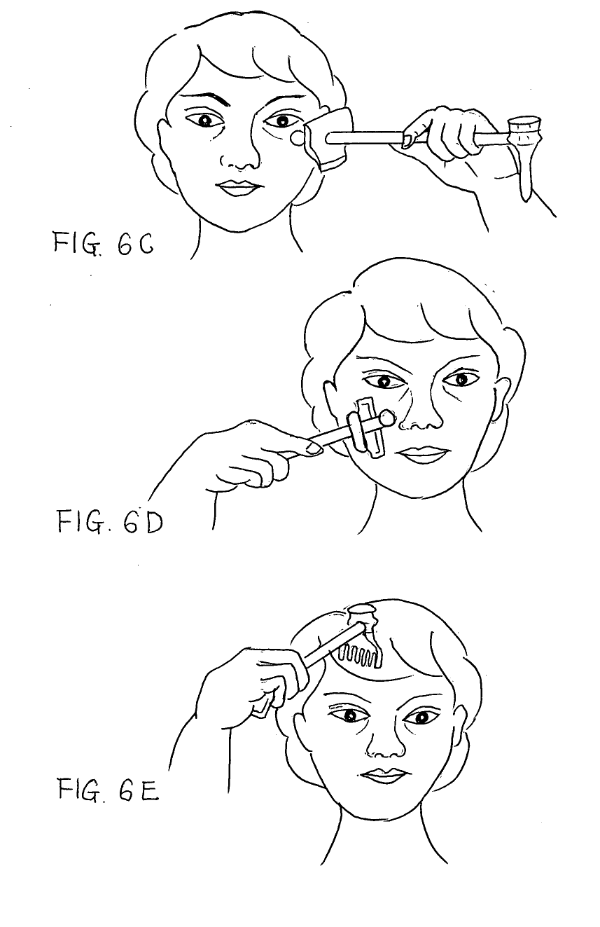

FIG. 6A shows use of the panel (4) of the device by an individual, such as a trained practitioner, on and around the pressure pain points on another individual's back by tapping, pressing, and/or rubbing. FIG. 6B shows use of the panel (4) of the device to tap, press, and/or rub on the pressure pain points on the foot.

In the case that an individual has pain on either his or her head or face including eyes, nose, mouth, and ears, instead of tapping with either the hammer head (2) or both the hammer head (2) and the pure silver nail together, it is better that pressing softly with the comb (3), the panel (4), or the outwardly extending protrusion (5) on and around the area of pain is performed for about 3 to 5 minutes and then rubbing with the outwardly extending protrusion (5), the teeth of the comb (3), or the edge or face of the thickness of the panel (4) on and around the area of pain for about 5 to 7 minutes. FIGS. 6C and 6D show use of the panel (4) and the outwardly extending protrusion (5) of the device to press and/or rub on the face of an individual. The panel (4) and/or the outwardly extending protrusion (5) can be used for facial massage for beauty by pressing and rubbing softly with the panel (4) and/or the protrusion (5) on the face. The comb (3) is also used for prevention of hair loss and dandruff and promotion of hair re-growth by combing the scalp of head in the one direction from the front to the back of the head. FIG. 6E shows use of the comb (3) of the device to comb on the scalp of the head of an individual.

All of the hammer head (2), the comb (3), the panel (4), and the protrusion (5) of the pure silver device can be used for tapping, pressing, and rubbing on the skin of the body. The hammer head (2) and the comb (3) are preferably used when either hard and strong stimulation or the treatment of pain is needed, but the panel (4) and the protrusion (5) are preferably used when either soft and little stimulation or a massage for either beauty or pain treatment on the face and head is needed.

The frequency of the treatment with the pure silver device depends on pain and symptoms of the patient and the bodily condition of the patient. If the patient has either chronic or severe pain and symptoms, twice a week treatment may be necessary. Otherwise, treatment is typically once a week, once every two weeks, or when needed

Before treating pain with the pure silver device, the treatment can be facilitated by putting the proper size and number of pure silver rings on the specific finger corresponding to the personal constitution of individuals themselves to harmonize the obstructed bio-energy.

Using abdominal diagnosis based on the basic theory of oriental medicine, the pure silver device and the method carry out the diagnosis and treatment of the root of pain on the abdomen, and thus the effect of treatment can be positively maximized and the recurrence of pain can be prevented.

Because silver has the highest thermal conductivity of any element and the highest electrical conductivity of any metal, silver promotes the stimulation of the pressure pain points. Pure silver effectively maximizes the highest thermal and electrical conductivity of silver. the pure silver device produces better stimulation of the pressure pain points without penetration into the skin of the human body. Therefore, the pure silver device is more effective in diagnosing and treating pain than other devices made out of any other material, even sterling silver and magnetic material. TABLE 2 shows the differentiations between pure silver therapy and other material therapy.

With regard to the method of diagnosing and treating pain with the pure silver device, pressing for both diagnosis and treatment, tapping, and rubbing do not necessarily have to be performed directly onto the skin of both the abdomen and the symptomatic areas of the body, but can also be performed onto the thin clothing covering on the skin of the both areas of the body without taking the clothing off because the effects to diagnose and treat pain in both cases are not even slightly different. Due to the highest thermal and electrical conductivity of pure silver, the pure silver device can still stimulate effectively the pressure pain points and make the produced electric pass easily into the skin on the pressure pain points through the thin clothing. Thus, the pure silver device can be easily used to diagnose and treat pain of individuals who feel uncomfortable for taking their clothing off in front of other persons.

In addition, the pure silver device is portable, monetarily valuable for personal property, and durable for the treatment of pain. The pure silver device and the method is simple, easy, and useful in that anyone can diagnose and treat themselves anytime and anyplace as well as without even the knowledge of basic eastern medicine such as meridians and acupuncture points. Due to the antibiotic effect of silver itself as well as no penetration into the skin, the pure silver device and the method are very safe without any risk of infection, side effects or other medical risks, so even children and seniors can get safely treated.

### Examples

### Example 1

A 21-year-old young woman had severe dysmenorrheal and indigestion when in school or at home. She had to take painkillers every day. Although she had taken acupuncture and magnetic therapy for a long time, her complained-of symptoms had not completely been eliminated. She had pressure pain points on the abdomen (KD16R/L, KD11R/L, ST30R/L, CV4, and CV5). Tapping with both the hammer head (2) and pure silver nail together of the pure silver device on each point of the pressure pain points was performed for 3 minutes, and then rubbing with the comb (3) and the panel (4) of the device on or around the pressure pain points was performed for 5 minutes. After this, treatment had been performed once a week for 5 weeks. The pain of the pressure pain points on her abdomen was completely eliminated, and the complained-of symptoms were also treated.

### Example 2

A 57-year-old man complained of indigestion, hair loss, and dandruff. He had constitutional indigestion for a long time, so he had taken both electric and magnetic acupuncture, and moxibustion therapy together for his indigestion, but the treatment did not work well for him. He recently had hair loss and dandruff. He had pressure pain points on his abdomen (CV12, ST25R, and KD16L). He got treatment on CV12, ST25R, and KD16L by tapping with the hammer head (2) of the pure silver device and pure silver nail together on those points and on his head and scalp by tapping, rubbing, and combing with the comb (3) of the device 4 times a week for 3 months. He said that after the treatment with the pure silver device, he had no problem of digestion, and also dandruff was completely treated and hair loss stopped.

### Example 3

A 6-year-old girl had the symptom of shaking her hands due to an emotional disorder. She was very shy. After she entered school, she suddenly started to uncontrollably shake her hands a lot. She therefore saw a doctor and took medicine for the symptoms. However, her symptoms were not cured. She had a pressure pain point around KD16L. After she got the treatment on the point around KD16L only once by tapping with the hammer head (2) of the device on the point for 4 minutes, her mother said that she did not have any symptom of shaking her hands uncontrollably.

### Example 4

A 42-year-old woman had hyperthyroidism and migraine and also lost her appetite. Although she went to hospital and took the medicine instructed by the hospital for hyperthyroidism, her migraine got worse without cure of her hyperthyroidism. She had pressure pain points on CV4, CV5, KD16R/L, KD11R/L, and ST30R/L. Those pressure pain points on her abdomen were released by tapping with both the hammer head (2) and the pure silver nail of the device on the points for three minutes and the pressure pain points on her head were released by tapping with the hammer head (2) and rubbing with the comb (3) of the device on her head for six minutes. She had received that treatment three times a week for one month. After the treatments, she said that she did not have hyperthyroidism or migraines anymore, so she stopped going to the hospital.

### Example 5

A 72-year-old man had diarrhea and pain in his right shoulder. He frequently had diarrhea for years and suddenly had pain in his right shoulder a couple of months ago. He had received electric acupuncture and magnetic acupressure therapy for six months, but he still endured diarrhea and right shoulder pain. He had pressure pain points on ST25R, and ST25L. He had received therapy with the pure silver device twice a week for two months by tapping with the hammer head (2) on ST25R and ST25L points and on the pressure pain points on his right shoulder and rubbing with the comb (3) and the panel (4) on the points on the right shoulder. He said that his right shoulder pain was relieved and diarrhea stopped.

### Example 6

A 45-year-old woman had indigestion and cold hands and feet. She took many different oriental herb medicines for one year. In addition, she received acupuncture, moxibustion, and magnet therapy for about four months. However, she said that she did not get better and those therapies were not effective for her. She had many pressure pain points on her abdomen (CV4, CV5, CV12, KD16R/L, ST25R/L, KD11R/L, ST30L/R, SP13R/L, and LR12R/L). She had received the pure silver therapy twice a week for two months by tapping with both the hammer head (2) of the device and pure silver nail together on each of those pressure pain points on her abdomen for about three to five minutes. After the treatment, she was very happy with the result that her indigestion was cured and her hands and feet became warm.

### Example 7

A 9-year-old boy was smart but could not pay attention in class and concentrate on studying, so he made a lot of mistakes in taking exams in school. His mother said that she gave him many well-known vitamins and oriental herbal medicine for his lack of attention, but she had not seen any improvement in his symptoms. He had pressure pain points on KD16R and KD16L. He had received the pure silver therapy once a week for two months by tapping with the hammer head (2) on the pressure pain points. Since then, her mother said that he was able to pay much better attention than before.

### Example 8

A 54-year-old woman had suffered from constipation for 20 years in that she defecated once every three or four days. She therefore drank water and vitamin drinks and ate certain foods for the treatment of constipation. She had the pressure pain points on CV12, ST25R, and ST25L. She had received the pure silver therapy once a week for 2 months by releasing the pressure pain points through tapping with the hammer head (2) of the device and pure silver nail together on the points. Since then, she could defecate once a day and wanted to keep taking this pure silver therapy.

### Example 9

A 36-year-old woman suffered from neck disk and pain in her shoulder. She had received massage therapy and acupuncture therapy for more than six months, but she still suffered from pain in her neck and shoulder. She had pressure pain points on her abdomen (CV4, CV5, KD16R, and KD16L). She had received the pure silver therapy once every two weeks for 4 months by tapping on the pressure pain points on her abdomen as well as on the pressure pain points on both her neck and shoulder on both sides and by rubbing with the panel (4) and pressing with the outwardly extending protrusion (5) on and around the points on her neck and shoulder for 15 minutes. Since then, she said that she felt much better in reducing pain in her neck and shoulder, and she stopped receiving massage therapy.

### Example 10

A 15-year-old boy suffered from chronic fatigue and headaches, which often affected his studies in school. He had pressure pain points on his abdomen (CV4, CV5, KD16L, and ST25R). He had received the pure silver therapy twice a week for three months by tapping with the hammer head (2) of the device and pure silver nail together on the pressure pain points on both his abdomen and head for 30 minutes and by rubbing with the panel (4) and pressing with the outwardly extending protrusion (5) on and around the points on the head for 6 six minutes. He said that after he received the first treatment, his headache was gone right away. He also said that since then, he did not suffer fatigue.

### Example 11

A 27-year-old man had irritable bowel symptoms. He said that he usually was very healthy, but he felt uncomfortable in his belly and often had either diarrhea or constipation. He drank a lot of water, but he suffered from an upset stomach and bowels. He had pressure pain points on KD16L and ST25R. He received the pure silver therapy once by tapping with the hammer head (2) of the device and pure silver nail together on the pressure pain points of KD16L and ST25R for 10 minutes and by rubbing on and around the points for 5 minutes. After the therapy, he was surprised at the result that he felt very comfortable in his belly and the symptoms of upset stomach and bowels disappeared right away.

### Example 12

A 50-year-old woman had severe lower abdominal pain and stomach aches. She had rested at home for one month since she could not work due to pain in her stomach and lower abdomen. She had too many pressure pain points on her entire abdomen detected by pressing with only soft force. I, the inventor, advised her to immediately go to the hospital to take medical exams such as endoscopy, since as a result of diagnosis with the pure silver device on her abdomen, she had so very deep and severe pain that she could not stand the pure silver therapy in spite of tapping very lightly on the pressure pain points. She went to hospital and took several medical exams. She received a diagnosis of cancer in her lymph nodes from the hospital. Fortunately, due to early detection of the cancer, she got better by receiving chemotherapy. She thanked me for the advice and diagnosis of her pain.

I claim:

1. a pure silver device as a whole made out of at least 99.9% fine silver for diagnosing and treating pain comprising:
an elongated handle having a top end and a bottom end;
a head connected to said top end having a first end and a second end, said first end being shaped to form a hammer head having no sharp protrusion, said second end being shaped to form a comb having teeth, said hammer head and said comb being opposed to each other;
said bottom end having an outwardly expending protrusion;
a panel vertically attached to between said bottom end and said outwardly expending protrusion.

2. The pure silver device according to claim 1, wherein said panel is vertically attached to the point of about 1 to about 3 cm away from said bottom end.

3. The pure silver device according to claim 1, wherein said panel is in the shape of having both corners of the top of rectangle is indented roundly.

4. The pure silver device according to claim 1, wherein said panel is in the shape of triangle, rectangular, trapezoid, ellipse, semicircle, quarter-circle or any combination thereof.

5. The pure silver device according to claim 1, wherein said outwardly expending protrusion is in the form of a bead, an egg, a rounded mass, a nipple, a lump, a bump, a projection, or any combination thereof.

6. A method of diagnosing pain comprising:
applying said outwardly expending protrusion of the pure silver device to press either on the skin of acupuncture points on abdominal area of human body or on the skin of all around abdominal area of the human body, which ranges from above the pelvis to below the ribs, inch by inch on all outward directions from the navel in order to find out all pressure pain points on the abdominal area and then to press on the skin of symptomatic areas of human body in order to find out pressure pain points on the symptomatic areas.

7. A method of claim 6, wherein pressing with said outwardly extending protrusion on the skin of the abdominal area to find out the pressure pain points on the abdominal area is performed by steps of a) applying a gentle force on a particular point of the skin of the abdominal area; b) if pressing with a gentle force there is no pain on the point, then applying a medium force on the point; c) if pressing with the medium force there is no pain on the point, then applying a firm force on the point; d) if pressing with the firm force there is no pain on the point, then moving either by an inch onto another point of the skin of the abdomen or onto another acupuncture point on the abdomen.

8. A method of claim 6, wherein pressing with said outwardly expending protrusion is performed onto the thin clothing covering on the skin of both the abdominal area and the symptomatic areas of the body instead of directly on the skin of the both areas of the body.

9. A method of treating pain comprising:
applying the pure silver device firstly to the pressure pain points on the skin of the abdominal area by tapping on the pressure pain points on the skin of the abdominal area with said hammer head, and secondly to the pressure pain points on the skin of the symptomatic areas by tapping on the pressure pain points on the skin of the symptomatic areas with said hammer head or said comb, by pressing on the pressure pain points on the skin of the symptomatic areas with said comb, said panel, or said outwardly expending protrusion, by rubbing on and around the pressure pain points on the skin of the symptomatic areas with said comb, said panel, or said outwardly expending protrusion, by combing on and around the pressure pain points on the scalp of the head of human body with said comb, or by combining thereof.

10. A method of claim 9, wherein the tapping on the skin of the pressure points on both the abdominal area and the symptomatic areas is performed with speed of about 60 to about 70 times a minute for about one to about three minutes.

11. A method of claim 9, wherein the tapping on the skin of the pressure points on both the abdominal area and the symptomatic areas with said hammer head of the pure silver device is performed along with a pure silver nail which has no sharp protrusion on its bottom by putting said pure silver nail vertically on the pressure pain points and then tapping on an upper top surface of said pure silver nail with said hammer head.

12. A method of claim 11, wherein the tapping with both said hammer head and said pure silver nail together is performed onto thin clothing covering on the skin of both the abdominal area and the symptomatic areas of the body instead of directly on the skin of the both areas of the body.
